(19) United States
(12) Patent Application Publication
(10) Pub. No.: US 2022/0401636 A1
(43) Pub. Date: Dec. 22, 2022

(54) CATHETER CUFF

(71) Applicant: Medtronic, Inc., Minnespolis, MN (US)

(72) Inventors: Sean A. McDermott, Weymouth, MA (US); Stephen W. Murray, Attleboro, MA (US)

(21) Appl. No.: 17/354,744

(22) Filed: Jun. 22, 2021

(51) Int. Cl. A61M 1/28; A61M 39/02

(52) U.S. Cl. CPC A61M 1/285; A61M 39/0208; A61M 2205/0205; A61M 2207/00; A61M 2039/0223

(57) ABSTRACT

In some examples, a catheter assembly includes a catheter including an elongated catheter body defining a lumen, a first cuff and a second cuff. The first cuff includes a first cuff body comprising a material configured to promote epidermal tissue growth around or within the first cuff and an adhesive configured to adhere the first cuff body to an outer surface of the elongated catheter body at a first cuff position selectable by a user. The second cuff includes a second cuff body comprising the material configured to promote epidermal tissue growth around or within the second cuff body and an adhesive configured to adhere the second cuff body to the outer surface of the elongated catheter body at a second cuff position selectable by the user. A distance between the first and second cuff positions is customizable by the user based on a patient.

### TECHNICAL FIELD

The present disclosure relates to medical catheters.

### BACKGROUND

A medical catheter defining at least one lumen has been proposed for use with various medical procedures. For example, a medical catheter may be used for dialysis, such as peritoneal dialysis (PD). During PD, a PD cycler delivers a dialysate through a catheter into a peritoneal cavity of a patient. The peritoneum of the patient acts as a membrane through which waste products are removed from the blood of the patient via osmosis and diffusion. Waste products and fluid pass from the blood of the patient, through the peritoneum, and into the dialysate. After a dwell period, the PD cycler removes an effluent fluid from the peritoneal cavity, which includes the dialysate and filtered waste products, through the catheter.

### SUMMARY

In examples described herein, a catheter includes at least one cuff configured to promote tissue ingrowth around the cuff, e.g., to help secure the catheter in place relative tissue of a patient. The position of the cuff along a longitudinal axis of the catheter is customizable by a user, e.g., during a medical procedure in which the catheter is introduced into a body of the patient, to enable the user to select a distance between a distal end of the catheter and the cuff and/or to select a distance between cuffs if the catheter includes multiple cuffs. The cuff includes a cuff body and an adhesive. The cuff body is formed from a material configured to promote growth of tissue and collagen on and around the cuff body material and anchor the cuff to the surrounding tissue and reduce or even prevent movement of the portion of a catheter internal to a patient due to movement of external portions of the catheter. The adhesive may be configured to adhere to both the cuff body and to an outer surface of the catheter.

In some examples, the catheter is a peritoneal dialysis catheter. In some cases, customizing a distance between distal openings of the catheter and the cuff may facilitate better peritoneal dialysis outcomes for the patient by enabling the catheter to be better fitted to the anatomy of the particular patient. For example, a distance between the cuff and the openings in a distal portion of the catheter may impact the extent to which the openings are open or closed, which can impact the efficiency of fluid introduction or removal from the peritoneal cavity of the patient.

In some example techniques, a user determines a cuff position along the catheter, at least partially wrap the cuff (e.g., cuff body and adhesive) around an outer perimeter of the catheter at the determined cuff position, and adheres the cuff to the catheter at the determined cuff position so as to customize the length between cuffs (if the catheter includes multiple cuffs) and/or the length from the cuff to a distal end of the catheter.

Clause 1: A catheter assembly including: a catheter including an elongated catheter body defining a lumen; a first cuff including: a first cuff body comprising a material configured to promote epidermal tissue growth around or within the first cuff; and an adhesive configured to adhere the first cuff body to an outer surface of the elongated catheter body at a first cuff position selectable by a user; a second cuff including: a second cuff body comprising the material configured to promote epidermal tissue growth around or within the second cuff body; and an adhesive configured to adhere the second cuff body to the outer surface of the elongated catheter body at a second cuff position selectable by the user, wherein a distance between the first and second cuff positions is customizable by the user based on a patient.

Clause 2: The catheter assembly of clause 1, wherein the elongated catheter body comprises one or more markings along a length of the elongated catheter body, wherein the one or more markings indicate a plurality of different catheter cuff positions selectable by the user.

Clause 3: The catheter assembly of clause 1 or 2, wherein the first and second cuff positions are based on a determined length between a subcutaneous cuff position and a deep cuff position of the patient.

Clause 4: The catheter assembly of any one of clause 1 through 3, wherein at least a distal portion of the elongated catheter body is coiled.

Clause 5: The catheter assembly of any one of clause 1 through 4, wherein the first and second cuff bodies have at least one of a keyed shape or a helical shape.

Clause 6: The catheter assembly of any one of clause 1 through 5, wherein the cuff body comprises an antimicrobial coating.

Clause 7: The catheter assembly of any one of clause 1 through 6, wherein the adhesive is configured to adhere to at least one of silicone, urethane, or polyurethane.

Clause 8: The catheter assembly of any one of clause 1 through 7, wherein the first and second cuff each further comprise a release liner, wherein the release liner is configured to releasably attach to the adhesive.

Clause 9: The catheter assembly of any one of clause 1 through 8, wherein the first cuff body and the second cuff body each comprise a polyester fabric and a primer disposed on the polyester fabric, the primer configured to promote adhesion between the adhesive and the polyester fabric.

Clause 10: The catheter assembly of any one of clause 1 through 9, wherein the adhesive is a pressure sensitive adhesive.

Clause 11: A catheter assembly including: a peritoneal dialysis catheter comprising an elongated catheter body defining a lumen, a distal portion of the elongated catheter body defining a plurality of sidewall openings open to the lumen and configured to at least one of introduce fluid into a peritoneal cavity of a patient or remove fluid from the peritoneal cavity; and a cuff comprising: a cuff body comprising a material configured to promote epidermal tissue growth around or within the cuff; and an adhesive configured to adhere the cuff body to an outer surface of the elongated catheter body at a cuff position selectable by a user.

Clause 12: The catheter assembly of clause 11, wherein the elongated catheter body comprises one or more markings along a length of the elongated catheter body, wherein the one or more markings indicate one or more of a plurality of different catheter cuff positions selectable by the user.

Clause 13: The catheter assembly of clause 11 or 12, wherein the cuff position is based on a determined length between a subcutaneous cuff position and a deep cuff position of the patient.

Clause 14: The catheter assembly of any one of clause 11 through 13, wherein the cuff body has at least one of a keyed shape or a helical shape.

Clause 15: The catheter assembly of any one of clause 11 through 14, wherein the cuff body comprises an antimicrobial coating.

Clause 16: The catheter assembly of any one of clause 11 through 15, wherein the adhesive is configured to adhere to at least one of silicone, urethane, or polyurethane.

Clause 17: The catheter assembly of any one of clause 11 through 16, wherein the cuff further comprises a release liner, wherein the release liner is configured to releasably attach to the adhesive.

Clause 18: A method of forming a peritoneal dialysis catheter, the method including: determining a first cuff position on the peritoneal dialysis catheter; wrapping a first cuff around a perimeter of an outer surface of the peritoneal dialysis catheter at the first cuff position, wherein the first cuff comprises: a first cuff body comprising a material configured to promote epidermal tissue growth around or within the first cuff; and an adhesive configured to adhere the first cuff body to an outer surface of the peritoneal dialysis catheter body at the first cuff position; adhering, via applying pressure to the first cuff, the adhesive to the outer surface of the peritoneal dialysis catheter; determining a second cuff position on the peritoneal dialysis catheter; wrapping a second cuff around the perimeter of the outer surface of the peritoneal dialysis catheter at the second cuff position, wherein the second cuff comprises: a second cuff body comprising a material configured to promote epidermal tissue growth around or within the second cuff; and an adhesive configured to adhere the second cuff body to an outer surface of the peritoneal dialysis catheter body at the second cuff position; and adhering, via applying pressure to the second cuff, the adhesive to the outer surface of the peritoneal dialysis catheter.

Clause 19: The method of clause 18, further comprising, before wrapping the first and second cuffs around the perimeter of the outer surface of the peritoneal dialysis catheter: removing a first release liner of the first cuff, the first release liner covering the adhesive of the first cuff; and removing a second release liner of the second cuff, the second release liner covering the adhesive of the second cuff.

Clause 20: The method of clause 18 or 19, wherein the peritoneal dialysis catheter comprises a plurality of markings along a length of the peritoneal dialysis catheter body, and wherein wrapping the first and second cuffs around the perimeter of the outer surface of the peritoneal dialysis catheter comprises aligning the first and second cuffs with respective markings of the plurality of markings.

The details of one or more examples are set forth in the accompanying drawings and the description below. Other features, objects, and advantages will be apparent from the description and drawings, and from the claims.

### BRIEF DESCRIPTION OF THE DRAWINGS

FIG. 1 is a diagram illustrating an example PD system configured to provide a patient with PD treatment.

FIG. 2A is a conceptual illustration of an example straight PD catheter that may be used in the PD system of FIG. 1.

FIG. 2B is a conceptual illustration of an example swan-neck PD catheter that may be used in the PD system of FIG. 1.

FIG. 2C is a conceptual illustration of an example straight PD catheter with a coiled intraperitoneal segment that may be used in the PD system of FIG. 1.

FIG. 2D is a conceptual illustration of an example PD swan-neck catheter with a coiled intraperitoneal segment that may be used in the PD system of FIG. 1.

FIG. 3 is a conceptual cross-sectional view of an example placement of one or more cuffs of a PD catheter within a patient.

FIG. 4A is a conceptual plan view of an example rectangular cuff.

FIG. 4B is a conceptual illustration of the rectangular cuff of FIG. 4A positioned, wrapped around, and attached to a catheter.

FIG. 5A is a conceptual plan view of an example quadrilateral-shaped cuff.

FIG. 5B is a conceptual illustration of the quadrilateral-shaped cuff of FIG. 5A positioned, wrapped around, and attached to a catheter.

FIG. 6A is a conceptual plan view of an example keyed cuff.

FIG. 6B is a conceptual illustration the keyed cuff of FIG. 6A positioned, wrapped around, and attached to a catheter.

FIG. 7A is a conceptual plan view of an example spiral cuff.

FIG. 7B is a conceptual illustration of the spiral cuff of FIG. 7A positioned, wrapped around, and attached to a catheter.

FIG. 8 is a cross-sectional view of an example cuff, where the cross-section is taken in a direction orthogonal to a longitudinal axis of the cuff.

FIG. 9 is a flow diagram of an example method of attaching a cuff to a catheter.

FIG. 10 is a flow diagram of another example method of forming a catheter.

### DETAILED DESCRIPTION

The present disclosure describes catheter cuffs, medical catheters including the cuffs, assemblies, techniques for making and using the catheter cuffs. In some examples, the cuffs are configured to be attached to a medical catheter (also referred to as a catheter herein) at a customizable position.

Some catheters, such as dialysis catheters, include one or more cuffs to promote tissue in-growth and stability, e.g., for chronic applications of a catheter. In existing catheters, cuffs are attached to the catheter at the manufacturing site in a fixed position. The cuff(s) may be fixed in position such that the distance between a cuff and the distal end of the catheter is fixed and/or non-variable. In addition, if the catheter includes multiple cuffs, the cuffs preattached to the catheter at the manufacturing site results in the distance between cuffs being fixed and/or non-variable. Both fixing the distance between a cuff and the distal end of the catheter and fixing the distance between multiple cuffs (if present) may make positioning of the distal end of the catheter in a patient more difficult and may make positioning of multiple cuffs in the patient more difficult.

Peritoneal dialysis (PD) catheters are used to access the peritoneal cavity of a patient to fill the peritoneal cavity with dialysate and to drain the dialysate. It may be desirable to position the PD catheter such that the distal end is within or near the pelvic gutter. If the distal end is too far down, the one or more sidewall openings near or at the distal end of the catheter may become plugged and may lead to patient discomfort, e.g., “drain pain.” If the distal end of the catheter is not far down enough into the peritoneal cavity, then the exchange of fluid and/or dialysate may not be able to be completed during a dialysis session. Further, there may be constraints on the exit and entry points of a patient for the catheter. For example, different patients have differing thicknesses of skin, fat, and other tissue, and a fixed distance between a subcutaneous cuff of the PDT catheter and a peritoneal wall cuff of the PD catheter may make it more difficult to find suitable exit/entry points on a particular patient.

The present disclosure describes a cuff configured to enable a user (referred to herein generally as a clinician) to select the position of one or more cuffs along the longitudinal length of a catheter. The cuff includes a cuff body and an adhesive. In some examples, the cuff further includes a release liner configured to releasably cover the adhesive until a user wishes to expose the adhesive, e.g. just prior to use. The adhesive is configured to attach the cuff body to a catheter and enables a clinician to position and adhere the cuff at one of a plurality of different longitudinal positions and/or locations of the catheter in order to vary and customize the distance between the cuff and the distal end of the catheter, e.g., to accommodate different patient anatomies or clinician preferences. In some examples, two or more cuffs may be attached to the same catheter, and the cuffs described herein enable a clinician to position and adhere two or more cuffs to the catheter in order to additionally vary and customize the distance between the cuffs, e.g., to accommodate different patient anatomies or clinician preferences.

The cuff body is formed from a material that is configured to promote and/or encourage growth of tissue and collagen, or epidermal tissue, on, around, and/or within the cuff. The growth of tissue and collagen on and around the cuff helps anchor the cuff to surrounding tissue and prevent movement of the portion of the catheter internal to the patient due to movement of external portions of the catheter, e.g., during coupling to PD cycler and/or during patient activity and movement. In addition, the growth of tissue and collage on and around the cuff further may help form a seal about the cuff to reduce and/or prevent infection, e.g., to prevent microbes from entering the patient. In some examples, the cuff body may be made of any suitable material configured to encourage the growth of tissue and collagen on and around the cuff, e.g., Dacron™, a polyester fabric, or a biocompatible porous material and the like.

The cuff body can have any suitable configuration that enables it to be attached to an outer surface of a catheter body of a catheter. In some examples, a cross-sectional shape of the cuff may be rectangular, a parallelogram, a quadrilateral, keyed, spiral, chiral, helical, or any suitable shape for wrapping around the outer perimeter of an outer surface of a catheter body. For example, the cross-sectional shape of the cuff body may be configured to reduce and/or minimize a circumferential gap between the ends of the cuff body to reduce and/or minimize a weak spot, e.g., less in-growth at or near such a gap. In some examples, the cross-sectional shape of the cuff body may be configured to increase the accuracy and ease of a user, such as a clinician, adhering the cuff body to the catheter with a reduced/minimized gap.

The adhesive is configured to have a suitable adhesion to the cuff body, or in some examples to a primer or other intermediate layer configured to promote adhesion between the cuff body and the adhesive. The adhesive may further be configured to have a suitable adhesion to an outside surface of the catheter, e.g., silicone, polyurethane, or any other suitable catheter material.

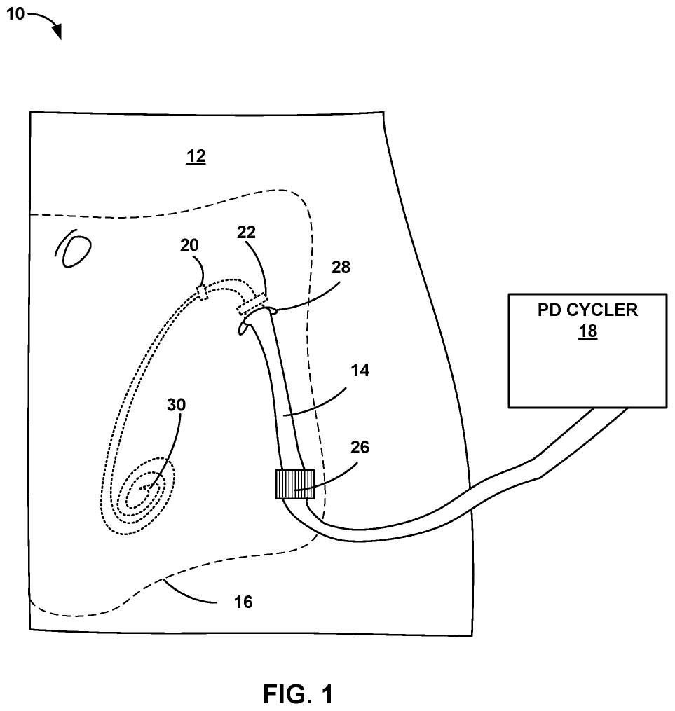

FIG. 1 is a diagram illustrating an example PD system 10 configured to provide a patient 12 with PD treatment. PD system 10 includes a catheter 14, cuffs 20, 22, and a PD cycler 18. Catheter 14 is illustrated in FIG. 1 as extending into a peritoneal cavity 16 of patient 12. Patient 12 ordinarily will be a human patient. In some cases, however, PD system 10 may be applied to other mammalian or non-mammalian, non-human patients.

PD cycler 18 is configured to deliver a dialysate into peritoneal cavity 16 via catheter 14. The dialysate remains in peritoneal cavity 16 for a dwell period, which has a duration sufficient for the exchange of waste products across a peritoneum of patient 12 to take place. In some such examples, PD cycler 18 may be disconnected from catheter 14 during the dwell period. In other examples, however, PD cycler 18 remains connected to catheter 14 during the dwell period. After the dwell period, PD cycler 18 removes fluid from peritoneal cavity 16. The fluid drained from peritoneal cavity 16 can be referred to as an effluent fluid, which contains the dialysate and the waste products removed from the blood of patient 12.

In some examples, the waste products may be removed by the dialysate due to a concentration gradient, e.g., due to the concentration of an osmotic agent in the dialysate, created in peritoneal cavity 16 when peritoneal cavity 16 is filled with the dialysate, which drives ultrafiltration and convective solute removal. The dialysate may include water, and dextrose or other sugars, salt, electrolytes, ions, and/or minerals as the osmotic agent. In some examples, the dialysate is dextrose-based, e.g., includes dextrose as the osmotic agent.

In order to deliver the dialysate into peritoneal cavity 16 and remove the effluent fluid from peritoneal cavity 16, PD cycler 18 is connected to catheter 14, such as via an adapter 26, which provides the necessary mechanical connection between catheter 14 and PD cycler 18 to establish fluid communication therebetween. Catheter 14 may be any fluid delivery conduit capable of being inserted into peritoneal cavity 16 and connected to PD cycler 18 to facilitate PD treatment of patient 12. Catheter 14 may be inserted into patient 12 via an exit site 28, and be configured to remain in patient 12 on a long-term basis, with a portion of catheter 14 remaining within peritoneal cavity 16 and a portion of catheter 14 residing outside of patient 12.

Catheter 14 have any suitable configuration. For example, the portion of catheter 14 that remains within peritoneal cavity 16 may be straight or curvilinear, such as pig-tailed (e.g., coiled). In some examples, a distal portion of catheter 14 has a swan neck (e.g., a curved portion up to about 180 degrees), which may help position catheter 14 at exit site 28 as intended. Catheter 14 has any suitable length for accommodating PD treatment. For example, catheter 14 may be between about 57 cm and about 62 cm in length (e.g., from adapter 26 to a distal-most end of catheter 14 within peritoneal cavity 16), and may be between about 2.5 mm and about 3.5 mm in diameter. In other examples, other shapes, sizes (e.g., length or diameter), and/or configurations may be used. An example of catheter 14 includes, but is not limited to, Argyle™ Peritoneal Dialysis Catheter available from Medtronic, Inc. of Minneapolis, Minn.

Cuffs 20, 22 may be configured to be attached to, and wrapped around, the outer perimeter of an outer surface of catheter 14 at one or more longitudinal positions along catheter 14. In the example shown in FIG. 1, one or both cuffs 20, 22 are located subcutaneously within patient 12. For example, in the example shown, cuff 22 is attached to catheter 14 at a longitudinal position of catheter 14 corresponding to, or near, exit site 28, and cuff 20 is attached to catheter 14 at a longitudinal position of catheter 14 corresponding to, or near, an internal exit site of patient 12, e.g., at or near the abdominal wall and/or the parietal peritoneum of patient 12.

In examples described herein, one or both cuffs 20, 22 are configured to be custom-positioned on catheter 14 by a clinician (or other user), rather than being prepositioned on catheter 14 at a manufacturing facility. For example, cuffs 20, 22 may initially be separate from catheter 14 and include an adhesive. A clinician may attach cuffs 20, 22 to catheter 14 at a clinician-selected longitudinal position of catheter 14. In some examples, a single cuff may be used positioned and attached to catheter 14, and in some examples two or more cuffs may be positioned and attached to catheter 14.

In some examples, to facilitate positive PD therapy outcomes, a clinician may select the locations at which one or both cuffs 20, 22 are positioned on catheter 14 based on a desired distance between the respective cuff 20, 22 and distal end 30 of catheter 14. For example, cuff 22 may be positioned and attached to catheter 14 based on a type of catheter 14, e.g., straight, coiled, swan-necked, and the like, and/or based on anatomy of patient 12. As another example, a length and/or size of the peritoneal cavity of patient 12 may vary from patient to patient, such that a desirable distance between cuff 22 and distal end 30 to facilitate effective PD therapy may vary based on the specific anatomy of patient. Thus, in some examples, a clinician can position and attach cuff 22 to catheter 14 based on patient 12 anatomy and/or patient 12 or clinician preference, e.g., so as to anchor catheter 14 via cuff 22 with a predetermined and/or desired distance between cuff 20 and distal end 30.

In some examples, a clinician may use a multiple cuff catheter 14, e.g., to further isolate the distal portion of catheter 14 within patient 12 from movement and/or forces on the proximal portion of catheter 14 outside of patient 12 and/or to potentially further reduce a risk of peritonitis. Accordingly, in some examples, in addition to a desired distance between one or both cuffs 20, 22 and distal end 30 of catheter 14, clinician may select the locations at which multiple cuffs 20, 22 are positioned on catheter 14 based on a desired distance between the cuffs 20, 22. For example, a factor a clinician may consider when selecting the relative locations of cuffs 20, 22 on catheter 14 is the locations of potential exit sites 28 and internal peritoneal cavity entry sites, which can vary based on patient anatomy and/or clinician preference. Thus, in some examples, a clinician can position and attach multiple cuffs 20, 22 to catheter 14 based on patient 12 anatomy and/or patient 12 or clinician preference, e.g., so as to achieve a predetermined and/or desired distance between cuffs 20, 22. This may provide a clinician with more freedom to select exit site 28 and/or an internal peritoneal cavity entry site, e.g., based on patient anatomy or clinician preference. That is, rather than selecting the exit site 28 and/or internal peritoneal cavity entry site to accommodate a predefined distance between cuffs of a catheter, the user-customizable cuffs described herein enable a clinician to first select the exit site 28 and/or internal peritoneal cavity entry site and then customize catheter 14 to accommodate the selected sites. This may result in better PD therapy outcomes in some cases because of better positioning of catheter 14 in patient 12.

As described herein, user-positionable cuffs 20, 22 may enable a clinician to customize the positioning of catheter 14 and/or exit site 28 and/or an internal peritoneal cavity entry site based on patient 12. In some examples, such as with single-cuff catheters, a cuff 20 or 22 may be positioned by the user and attached to catheter 14. For example, a clinician may determine a subcutaneous cuff position of catheter 14 for a subcutaneous cuff based on patient 12 anatomy, and cuff 22 may be positioned and attached at a longitudinal position of catheter 14 corresponding to cuff 22 being located subcutaneously near exit site 28 of patient 12 as illustrated. In another single-cuff catheter example, a clinician may determine a deep cuff position of catheter 14 for a deep cuff based on patient 12 anatomy, and cuff 20 may be positioned and attached at a longitudinal position of catheter 14 corresponding to cuff 20 being located at or near an internal peritoneal cavity entry site of patient 12.

Although a catheter including two cuffs 20, 22 is shown and described with reference to FIG. 1, in other examples, only one cuff 20 or 22 can be attached to catheter 14, or more than two cuffs can be attached to catheter 14 using the example cuffs and techniques described herein.

Cuffs 20, 22 may be configured to promote and/or encourage growth of tissue, collagen, or epidermal tissue on, around, or within each of cuffs 20, 22, e.g., cuffs 20, 22 may include a cuff body material configured to promote and/or encourage growth of tissue, collagen, or epidermal tissue on, around, or within cuffs 20, 22. In some examples, cuffs 20, 22 may comprise a biocompatible porous material, such as a polyester fabric, Dacron™, and the like. In some examples, cuffs 20, 22 may include an antimicrobial coating, e.g., the cuff body material may include an antimicrobial coating.

Cuffs 20, 22 further include an adhesive to facilitate attachment to catheter 14 at user-selected positions. For example, an adhesive may be disposed on a major surface of cuffs 20, 22, e.g., attached, laminated, coated, and the like. In some examples, cuffs 20, 22 may include a primer and/or primer layer between the cuff body material and the adhesive, e.g., to promote adhesion between the cuff body material and the adhesive and/or to planarize a major surface of the cuff body material for better adhesion of the adhesive to the cuff body material and/or to be a barrier between the adhesive and the cuff body material to prevent the adhesive from absorbing within the cuff body.

In some examples, the adhesive is configured to adhere to the cuff body material of cuffs 20, 22 and/or a primer, and to adhere to an outer surface of catheter 14. The adhesive can be configured to be activated on site at the clinic, e.g., just prior to use, and without any special equipment, e.g., to cure the adhesive. For example, the adhesive may be configured to adhere to silicone, urethanes, polyurethanes, and/or any other suitable material comprising the outer surface of catheter 14. In some examples, the adhesive may be a silicone adhesive, a pressure sensitive adhesive (PSA), a silicone PSA, or any other suitable adhesive for adhering to catheter 14.

Cuffs 20, 22 may include a release liner. For example, cuffs 20, 22 may include a release liner configured to cover and/or protect the adhesive, and be removable/releasable from the adhesive. In some examples, the release liner may be made of a polymer, paper, or any suitable release liner material. In some examples, the release liner may include one or more coatings configured for an adequate adhesion to the adhesive, e.g., at least a minimum amount of adhesion to protect and stay on the adhesive during transport and handling of cuffs 20, 22 and less than a maximum amount of adhesion to allow the release liner to be removed by a user and/or clinician, e.g., relatively easily and without damage to the cuff body material and/or the adhesive. For example, the release liner may be configured to be removable from the adhesive without the adhesive detaching, removing, or peeling from the cuff body material (or primer) of cuffs 20, 22.

Cuffs 20, 22 have a length that is selected to enable cuffs 20, 22 to extend around an entire or a majority of an outer perimeter of catheter 14, where the length is measured in a direction orthogonal to a longitudinal axis of catheter 14 when cuffs 20, 22 are attached to catheter 14. That is, a length of cuffs 20, 22 is based on an outer perimeter (e.g., a circumference) of catheter 14. For example, cuffs 20, 22 may be configured to wrap around the full outer perimeter of catheter 14 at least one time (e.g., one, two, three, or more times). In some examples, cuffs 20, 22 have a length that is between 10 millimeters (mm) to 30 mm, 10 mm to 50 mm, or 5 mm to 100 mm. In some examples, cuffs 20, 22 have a width that is between 1 mm to 3 mm, 0.5 mm to 5 mm, or 0.1 mm to 10 mm. In some examples, each cuff 20, 22 defines opposite ends (referred to herein as the first and second ends), and the length of the cuff is measured between the ends. In some examples, the length of the respective cuff 20, 22 is selected such that the first and second ends of cuffs 20, 22 are configured to abut each other upon being wrapped around the outer perimeter of catheter 14, e.g., so as to reduce, minimize, and/or eliminate a gap between the first and second ends. A gap between the first and second ends may result in less tissue and/or collagen growth around the respective cuff, which can result in a weak spot that may be more susceptible to detaching from patient 12.

Cuffs 20, 22 may have any suitable width, where the width is measured in a direction orthogonal to the direction in which the length is measured. In some examples, cuffs 20, 22 may have a width that is less than the length between exit site 28 and or an internal peritoneal cavity entry site of patient 12.

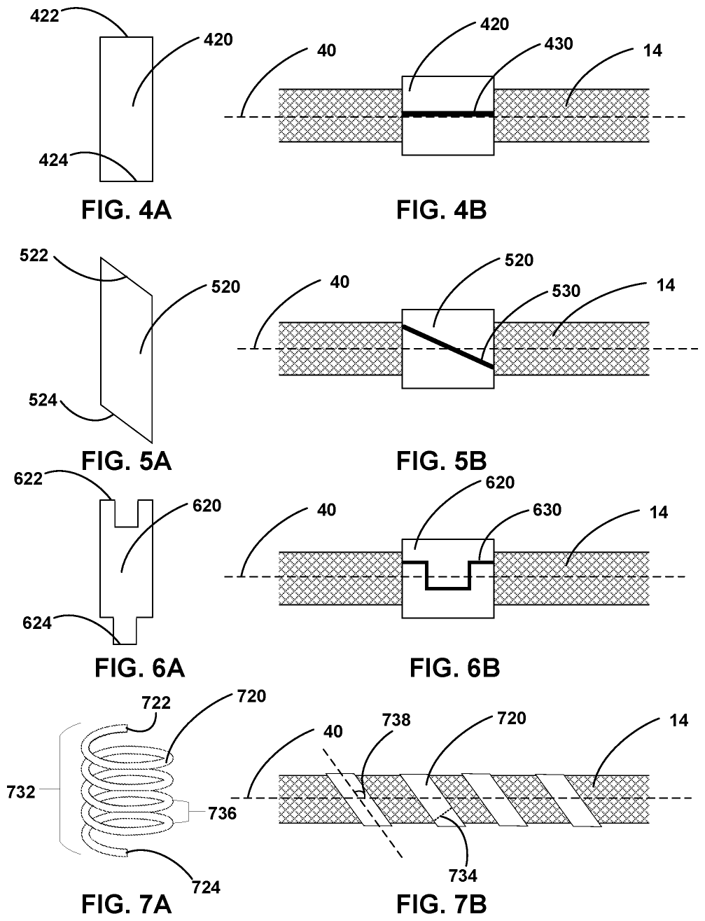

Cuffs 20, 22 may have any suitable shape that facilitate extending around an outer perimeter of catheter 14. In some examples, cuffs 20, 22 may have a keyed shape, a helical shape, a rectangular shape, a tapered end shape, a parallelogram shape, a quadrilateral shape, or any suitable shape, as further illustrated and described below with reference to FIGS. 4A-7B. In some examples, the shape of cuffs 20, 22 may determine the angle, shape, and/or length of a seam between the first and second ends of cuffs 20, 22, e.g., where the first and second ends abut. In some examples, overlap of the first and second ends of cuffs 20, 22 may be detrimental to the fit of cuffs 20, 22 within an exit/entry site and may cause and/or allow a “tunnel track” or void to form, rather than a complete in-growth of tissue, which may allow particles and/or microbes to enter patient 12, at least partially, via the tunnel track. The shape of cuffs 20, 22 may be configured to reduce overlap and/or ease alignment of the first and second ends of cuffs 20, 22 so as to reduce overlap. In some examples, the angle, shape, and/or length of a seam between the first and second ends of cuffs 20, 22 may determine an amount of tissue and/or collagen growth proximate to the seam and may determine whether there is a weak spot and how weak the weak spot may be. In some examples, the first and second ends of a respective cuff 20, 22 are not configured to abut, e.g., catheter cuffs 20, 22 with a helical shape where cuffs 20, 22 wrap around the outer surface of catheter 14 more than one time and the first and second ends are longitudinally and/or axially separated from each other along catheter 14, as illustrated below in FIG. 7B.

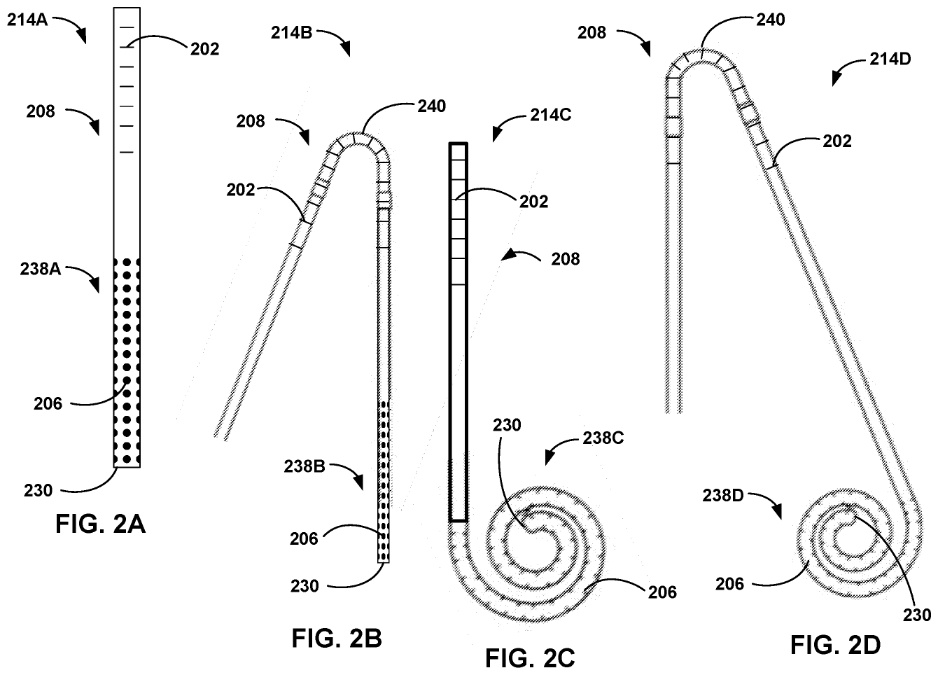

In some examples, PD catheter 14 includes one or more sidewall openings along a distal portion of catheter 14, where the openings are configured to enable fluid flow from an inner lumen of catheter 14 into and out of PD cavity 16. FIGS. 2A-2D are conceptual illustrations of example PD catheters, which are examples of catheter 14 of FIG. 1. One or both cuffs 20, 22 can be attached to any of the catheters shown in FIGS. 2A-2D in various examples. FIGS. 2A-2D are described concurrently below. FIG. 2A is a conceptual illustration of an example straight (e.g., Tenckhoff) PD catheter 214A that may be used in the PD system of FIG. 1. FIG. 2B is a conceptual illustration of an example swan-neck catheter 214B that may be used in the PD system of FIG. 1. FIG. 2C is a conceptual illustration of an example catheter 214C with a coiled intraperitoneal segment that may be used in the PD system of FIG. 1. FIG. 2D is a conceptual illustration of an example swan-neck catheter 214D with a coiled intraperitoneal segment that may be used in the PD system of FIG. 1.

In the example shown in FIGS. 2A and 2B, catheters 214A and 214B include a straight distal portion 238A, 238B, respectively, that is configured to be positioned within peritoneal cavity 16 when catheter 214A, 214B is used to provide patient 12 with PD. In other examples, however, the distal portion can have another suitable shape, e.g., coiled or a different curvilinear shape. For example, catheters 214C and 214D include a coiled (e.g., pigtailed) distal portion 238C, 238D, respectively. Additionally, catheters 214B and 214D are shown to have a preformed bend 240, which may help decrease stress on exit site 28 (FIG. 1) during use of catheters 214B, 214D.

In the examples, shown, catheters 214A-214D include one or more sidewall openings 206 along at least a portion of the length of distal portions 238A-238D. The one or more sidewall openings 206 are configured to introduce fluid from a lumen of the respective catheter into a peritoneal cavity of patient 12 or remove fluid from the peritoneal cavity of patient 12, or both.

In the examples shown, catheters 214A-214D including one or more markings 202 along a length of the elongated catheter body 208 of each of catheters 214A-214D. Markings 202 indicate one or more of a plurality of different catheter cuff positions selectable by a clinician (or other user). In some examples, markings 202 may be guides for positioning and attaching cuffs 20, 22 to catheters 214A-214D, e.g., so as to customize the length between distal end 230 and a cuff 20 or 22 and/or to customize the length between cuffs 20, 22. In some examples, markings 202 may be configured to guide a clinician as to where to position and adhere cuffs 20, 22 longitudinally along any of catheters 214A-214D. In some examples, markings 202 may be regularly spaced and/or irregularly spaced, and may be close enough to provide enough resolution in order to position cuffs 20, 22 with adequate resolution, e.g., to within 10 mm, 1 mm, 0.1 mm, or any suitable length resolution. In some examples, markings 202 may be configured to indicate the exact position for cuffs 20, 22, and in other examples, markings 202 may be configured as guides for positioning cuffs 20, 22. Markings 202 may be any type of marking, e.g., printed, recesses, bumps, stickers, or any suitable marking type visible to a user.

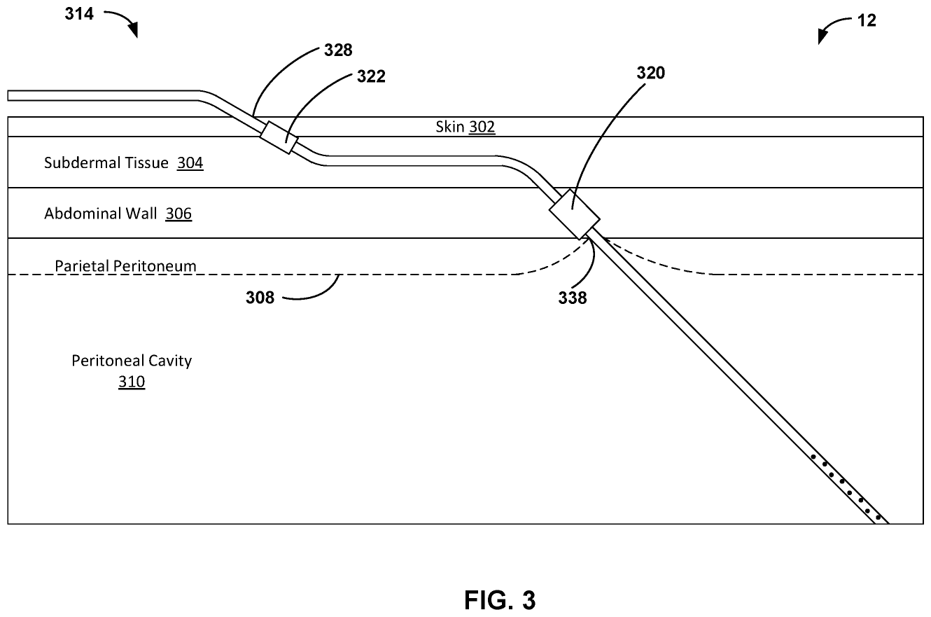

FIG. 3 is conceptual cross-sectional view of an example placement of one or more cuffs 320, 322 of a PD catheter 314 within a patient 12. In the example shown, catheter 314 is a double-cuff catheter placed within patient 12, and cuffs 320, 322 have been positioned and attached to catheter 314. Catheter 314 is an example of catheter 14 of FIG. 1 and cuffs 320, 322 are examples of cuffs 20, 22, respectively, of FIG. 1.

In some examples, the desired length between cuff 320 and cuff 322 may be based on anatomy of patient 12, where the length is measured along a longitudinal axis of catheter 314. For example, a clinician may determine the positions of, and the length between, cuffs 320, 322 based on the thicknesses of skin 302, subdermal tissue 204, and the abdominal wall 306 at or near exit site 328 and peritoneal cavity entrance site 338, as well as a length of catheter 314 that a clinician determines should run within any and/or all of skin 302, subdermal tissue 204, and abdominal wall 306. In some examples, cuff 322 may be positioned so as to be subcutaneous and proximate to skin 302, and cuff 320 may be positioned to be a “deep” cuff and/or a peritoneal cuff at or near an inner surface of abdominal wall 306 and/or the parietal peritoneum 308.

In some examples, the clinician may select the positions of one or both cuffs 320, 322 further based on the length between cuff 320 and/or cuff 322 and the distal end of catheter 314 (not shown), e.g., which may be based on the locations of exit site 328 and/or peritoneal cavity entrance site 338 and the length and/or size of peritoneal cavity 310 of patient 12. For example, the clinician may select the positions of cuffs 320, 322 such that sidewall openings of catheter 314 do not collapse, compress, or otherwise become obstructed to fluid flow within peritoneal cavity 310. The clinician may select the positions of cuffs 320, 322 such that the distal end of catheter 314 is not too long between peritoneal cavity entrance site 338 and the bottom of the pelvic gutter and thereby avoid and/or reduce the chance of the distal end of catheter 314 at least partially falling over within peritoneal cavity 310 causing the sidewall openings to become plugged, and resulting in drain pain. Additionally, the clinician may select the positions of cuffs 320, 322 such that the distal end of catheter 314 is not too short between peritoneal cavity entrance site 338 and the pelvic gutter to facilitate fluid removal, e.g., and avoid incomplete exchange of fluids via catheter 314.

In some examples, a user and/or clinician may determine the locations of exit site 328 and/or peritoneal cavity entrance site 338 based on patient 12 anatomy. For example, a clinician, user, and/or patient 12 may wish to avoid folds in skin 302 and/or the abdominal area of patient 12. The clinician may determine one or more desired exit sites 328, determine a length that catheter 314 should run within any and/or all of skin 302, subdermal tissue 204, and the abdominal wall 306, which may be based on a desired peritoneal cavity entrance site 338, determine a length of the distal portion of catheter 314 for adequate and/or optimal PD, e.g., such that catheter 314 is positioned correctly within peritoneal cavity 310 (e.g., so as to avoid positioning catheter 314 with a distal portion that is too long or too short within peritoneal cavity 16 of patient 12, as described above), and the position and attach cuffs 320, 322 accordingly based on those determinations.

FIGS. 4A-7B illustrate conceptual cross-sectional views of example cuff shapes before and after positioning and attaching to a catheter, e.g., catheter 14. In some examples, the cuff shape may improve adhesion and/or anchoring of the cuff to patient 12. For example, cuff shape may improve tissue and collagen growth and reduce weak spots of reduced tissue and or collage growth, e.g., where a weak spot may be a volume with reduced adhesion and/or anchoring to the tissue of patient 12 and or a reduced barrier to microbes or other matter or particles from entering patient 12.

FIG. 4A is a conceptual plan view of an example rectangular cuff 420, and FIG. 4B is a conceptual illustration of rectangular cuff 420 of FIG. 4A positioned, wrapped around, and attached to an outer surface of catheter 14. In the example shown, the first and second ends 422, 424 of rectangular cuff 420 abut to form seam 430 which is substantially straight and substantially parallel to the longitudinal axis 40 of catheter 14.

FIG. 5A is a conceptual plan view of an example quadrilateral cuff 520, and FIG. 5B is a conceptual illustration of quadrilateral cuff 520 of FIG. 5A positioned, wrapped around, and attached to an outer surface of catheter 14. In the example shown, the first and second ends 522, 524 of quadrilateral cuff 520 abut to form seam 530 which is substantially straight and at an angle with respect to the longitudinal axis 40 of catheter 14. In some examples, the quadrilateral shape of cuff 520 may provide an improved ease of alignment, abutment, and reduced overlapping of first and second ends 522, 524 when attaching of cuff 520 to catheter 14, e.g., relative to cuff 420.

FIG. 6A is a conceptual plan view of an example keyed cuff 620, and FIG. 6B is a conceptual illustration of keyed cuff 620 of FIG. 6A positioned, wrapped around, and attached to an outer surface of catheter 14. In the example shown, the first and second ends 622, 624 of keyed cuff 620 abut to form seam 630 which has a substantially rectangular shape along the longitudinal and/or axial direction of the length of catheter 14. The shape of the first and second ends of keyed cuff 620 may be any suitable shape. In the example shown, the shape of the first and second ends 622, 624 of keyed cuff 620 is rectangular, e.g., one or more periods of a rectangular function, wave, pattern, and the like. In other examples, the first and second ends 622, 624 of keyed cuff 620 may have a sawtooth, triangular, undulating (e.g., sinusoidal), or any other suitable type of pattern. The shape and/or pattern of seam 630 may be any suitable pattern, and corresponds to the shape of the first and second ends 622, 624 of keyed cuff 620, e.g., seam 630 may be a rectangular, sawtooth, triangular, sinusoidal, or any other suitable pattern. This enables first and second ends 622, 624 of cuff 620 to mate together, which may provide an improved ease of alignment, abutment, and reduced overlapping of first and second ends 622, 624 when attaching of cuff 620 to catheter 14, e.g., relative to cuff 420 and/or cuff 520.

FIG. 7A is a conceptual plan view of an example helical cuff 720, and FIG. 7B is a conceptual illustration of helical cuff 720 of FIG. 7A positioned, wrapped around, and attached to an outer surface of catheter 14. In the example shown, the first and second ends 722, 724 of helical cuff 720 do not abut and do not form a seam. The length 732, width 734, pitch 736, and angle 738 of helical cuff 720 may be any suitable length, width, pitch and angle. In the example shown, the length 732, pitch 736, and angle 738 of helical cuff 720 are such that helical cuff 720 wraps around catheter 14 at least 3.5 times, and additionally the width 734 is such that there is a space between adjacent wraps. In some examples, helical cuff 720 may not have a space between adjacent wraps around catheter 14, e.g., the sides and/or edges of adjacent wraps of helical cuff 720 abut and form a helical seam about the outer surface of catheter 14. In some examples, helical cuff 720 wraps around catheter 14 more or fewer times than 3.5 times.

FIGS. 4A-7B are exemplary, and in other examples, cuffs 20, 22 may have shapes corresponding to cuffs 420-720 or may have any other suitable shape. In some examples, cuffs 20, 22 may only partially wrap around the outer perimeter and/or circumference of catheter 14. In some examples, two or more cuffs may be positioned and attached to a catheter, and the two or more cuffs may be the same type, e.g., all cuffs may be cuff 720, or different from each other, e.g., a first cuff 420 and a second cuff 720.

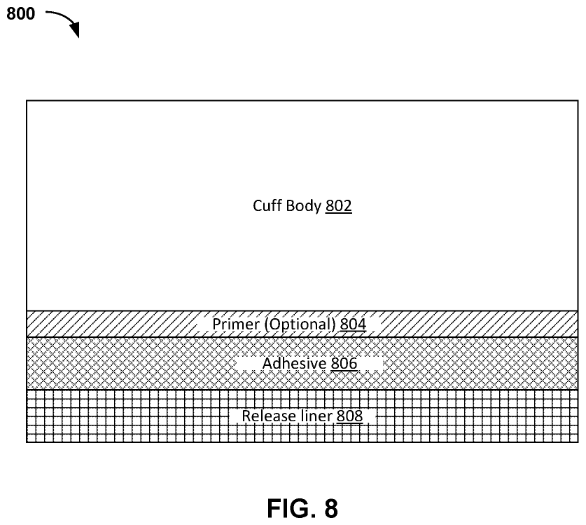

FIG. 8 is a cross-sectional view of an example cuff 800, where the cross-section is taken in a direction orthogonal to a length and width of cuff 800 and shows a thickness of cuff 800. Cuff 800 includes cuff body 802, optional primer 804, adhesive 806, and release liner 808. Cuff 800 may be an example of any cuff described herein, e.g., cuffs 20, 22 of FIG. 1, cuffs 320, 322 of FIG. 3, or cuffs 420-720 of FIGS. 4A-7B.

Cuff body 802 may be formed of a material that is configured to promote and/or encourage growth of tissue and collagen, or epidermal tissue, on, around, and/or within cuff 800. The growth of tissue and collagen on and around cuff 800 serves to anchor cuff 800 to surrounding tissue and prevent movement of the portion of the catheter internal to the patient due to movement of external portions of the catheter, e.g., during coupling to PD cycler and/or during patient activity and movement. The growth of tissue and collage on and around cuff 800 further serves to form a seal about cuff 800 to reduce and/or prevent infection, e.g., to prevent microbes from entering the patient. In some examples, cuff 800 body may be made of any suitable material configured to encourage the growth of tissue and collagen on and around cuff 800, e.g., Dacron™, a polyester fabric, or a biocompatible porous material and the like.

Cuff body 802 can have any suitable configuration that enables it to be attached to an outer surface of a catheter body of a catheter. In some examples, a cross-sectional shape of cuff body 802 may be rectangular, a parallelogram, a quadrilateral, keyed, spiral, chiral, helical, or any suitable shape for wrapping around the outer perimeter of an outer surface of a catheter body. For example, the cross-sectional shape of cuff body 802 body may be configured to reduce and/or minimize a circumferential gap between the ends of cuff body 802 body to reduce and/or minimize a weak spot, e.g., less in-growth at or near such a gap. In some examples, the cross-sectional shape of cuff body 802 body may be configured to increase the accuracy and ease of a user, such as a clinician, adhering cuff body 802 body to the catheter with a reduced/minimized gap.

Primer 804 may be configured to promote adhesion between cuff body 802 and adhesive 806, and may be disposed on cuff body 802 and between cuff body 802 and adhesive 806. In some examples, cuff 800 may not include primer 804. In some examples, cuff 800 may not include primer 804 and adhesive 806 may include a primer and/or primer material. In some examples, primer 804 is configured to adhere to both cuff body 802 and adhesive 806 (or a primer material, e.g., within adhesive 806), or is configured to cause adhesive 806 to adhere to cuff body 802 with an adhesion that is greater than the adhesion between adhesive 806 and release liner 808. In some examples, primer 804 may be configured to planarize a major surface of cuff body 802 for better adhesion of adhesive 806 to cuff body 802 and/or to be a barrier between adhesive 806 and cuff body 802, e.g., to prevent adhesive 806, or any other material such as water, moisture, oils, and the like, from absorbing within cuff body 802.

Adhesive 806 is configured to adhere and/or attach cuff body 802 to an outer surface of a catheter, e.g., PD catheter 14 of FIG. 1. Adhesive 806 enables a user to position and adhere and/or attach cuff 800 at one of a plurality of different longitudinal positions and/or locations of the catheter in order to vary and customize the distance between cuff 800 and the distal end of the catheter, e.g., to accommodate different patient anatomies or clinician preferences, and/or to vary and customize the distance between two or more cuffs.

Adhesive 806 may be configured to have a suitable adhesion to cuff body 802, or in some examples, to primer 804. Adhesive 806 may further be configured to have a suitable adhesion to an outside surface of the catheter, e.g., silicone, polyurethane, or any other suitable catheter material. In some examples, adhesive 806 may be a silicone adhesive, a pressure sensitive adhesive (PSA), a silicone PSA, or any other suitable adhesive for adhering to an outer surface of a catheter. Adhesive 806 may be disposed on a major surface of cuff body 802 (or primer 804), e.g., attached, laminated, coated, and the like.

Release liner 808 may be configured to cover and/or protect adhesive 806 until a user wishes to expose adhesive 806, e.g. just prior to use. Release liner 808 may be configured to releasably attach to adhesive 806. Release liner 808 may be configured to be removable from adhesive 806, e.g., with a sufficiently low adhesion such that a user may remove release liner 808 with relative ease and without causing adhesive 806 to detach, remove, or peel from cuff body 802 (or primer 804). In some examples, release liner 808 may be made of a polymer, paper, or any suitable release liner material. In some examples, release liner 808 may include one or more coatings configured for an adequate adhesion to adhesive 806, e.g., at least a minimum amount of adhesion to protect and stay on the adhesive during transport and handling of cuff 800 and less than a maximum amount of adhesion to allow the release liner to be removed by a user and/or clinician relatively easily and without damage to cuff body 802 and/or adhesive 806.

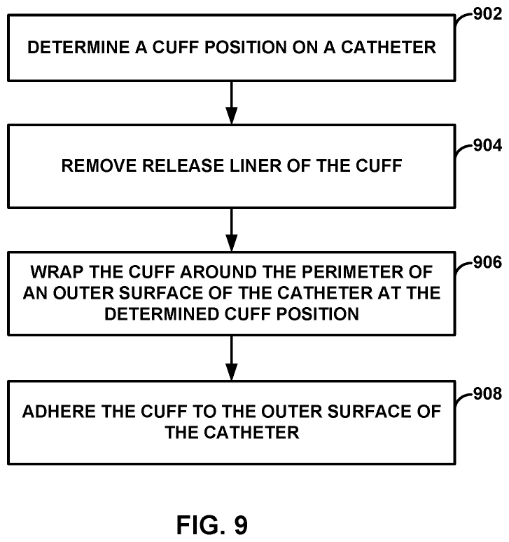

FIG. 9 is a flow diagram of an example method of forming a catheter, such as a PD. Although the example technique of FIG. 9 is described with respect to catheters 214A-214D of FIG. 2, patient 12 of FIG. 3, and cuff 800, the example technique of FIG. 9 may be performed to form any type of catheter that includes a cuff and any type of cuff described herein. In some examples, the technique of FIG. 9 may be performed by any suitable user, such as a clinician.

A user determines a cuff position on a catheter (902). In some examples, a user may determine one or more cuff positions based on a determination of the locations of exit site 328 and/or peritoneal cavity entrance site 338, which may each be based on anatomy of patient anatomy, such as the thickness of tissue layers between a skin surface of patient 12 and the peritoneal cavity 310 (FIG. 3). For example, the user may determine a peritoneal cavity entrance site 338 based on a length of the distal portion of catheter 314 and a size of peritoneal cavity 310 (which can be an approximate or measured size). As an example, the user may select peritoneal cavity entrance site 338 such that once catheter 314 is positioned within peritoneal cavity 310, the distal portion of catheter 314 is not kinked, bent, or otherwise positioned to block fluid flow through to sidewall openings 206 (FIG. 2A-2D) in the distal portion. The user may then determine a position of one or more cuffs 800 (e.g., as a deep cuff 320) accordingly based on that determined length. The clinician, user, and/or patient 12 may additionally wish to avoid folds in skin 302 and/or the abdominal area of patient 12, and the user may then determine one or more desired exit sites 328 based on any patient 12 feature including avoiding skin folds. The clinician and or user may then determine a length that catheter 314 should run within any and/or all of skin 302, subdermal tissue 204, and the abdominal wall 306 between peritoneal cavity entrance site 338 and exit site 328. The user may then determine a positions of one or more cuffs 800 (e.g., a subcutaneous cuff 322 at exit site 328) accordingly based on those determinations, e.g., the length of the distal portion of the catheter 314 for adequate and/or optimal PD and a determined peritoneal cavity entrance site 338 and the length that catheter 314 should run between the determined peritoneal cavity entrance site 338 and one or more desired exit sites 328.

In some examples, the user determines only one cuff position, e.g., a subcutaneous cuff position, a deep cuff position, or any other cuff position, each of which may be based on the lengths described above and/or exit site 328 and peritoneal cavity entrance site 338 or based other criteria, e.g., which may be patient-specific. In some examples, the user may determine the position one or more cuffs 800 using markings on the catheter, such as markings 202. The user may determine the position for a cuff exactly on one or more marking 202, or the user may determine the position of one or more cuffs 800 using markings 202 as a guide, e.g., for later positioning and attaching of the one or more cuff at the determined positions.

The user may remove a release liner of the cuff (904). For example, the user may remove release liner 808 to expose adhesive 806, e.g. just prior to wrapping and adhering cuff 800 to the catheter.

The user may wrap the cuff 800 around a perimeter of an outer surface of catheter 314 at the determined cuff position (906). For example, the user may wrap cuff 800 at least partially around the perimeter of an outer surface of catheter 314, or the user may wrap cuff 800 fully around the perimeter of the outer surface such that the ends of cuff 800 abut. In some examples, the user may wrap cuff 800 at least partially around, or fully around the perimeter of an outer surface of catheter 314 one or more times such that ends of cuff 800 do not abut, e.g., the user may wrap a helical cuff 800 (e.g., cuff 720 shown in FIGS. 7A and 7B) more than once around the perimeter of an outer surface of catheter 314. In some examples, catheter 14 may include a plurality of markings along a length of the PD catheter body, and the user may wrap cuff 800 around the perimeter of the outer surface of catheter 314 by aligning cuff 800 with respective markings of the plurality of markings, e.g., markings 202.

The user may adhere cuff 800 to the outer surface of catheter 314 (908). For example, the user may apply pressure to cuff 800 that is wrapped around the perimeter of the outer surface of catheter 314 at the determined cuff position. In some examples, the pressure may be a radially inward force that may cause adhesive 806 to adhere and/or increase adhesion to the outer surface of the catheter, e.g., adhesive 806 of cuff 800 may be a PSA that the user presses to the outer surface of the catheter. In these examples, no further specialized tools (e.g., light to cure an adhesive) may be needed to secure cuff 800 to catheter 314.

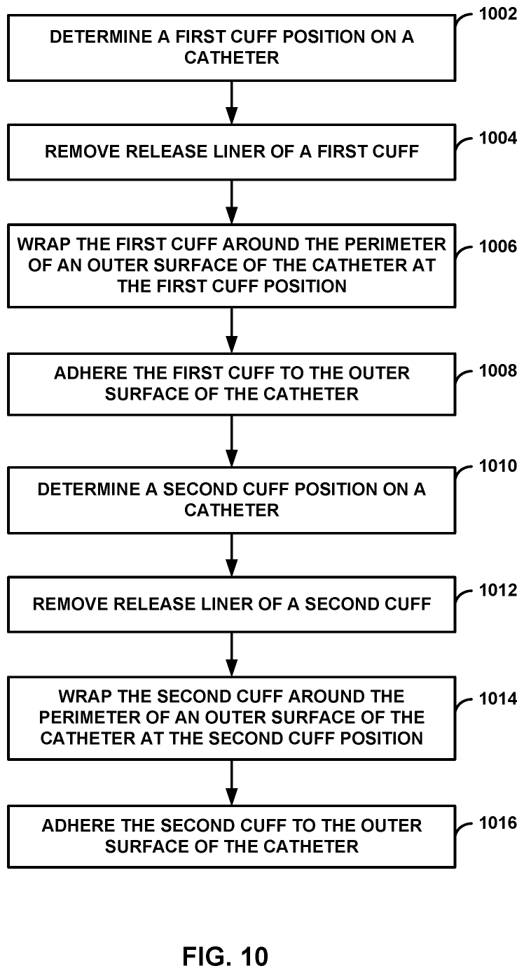

FIG. 10 is a flow diagram of another example method of forming a catheter. Although the example technique of FIG. 10 is described with respect to catheters 214A-214D of FIG. 2, patient 12 of FIG. 3, and cuff 800, the example technique of FIG. 10 may be performed to form any type of catheter that includes a cuff and any type of cuff described herein. In some examples, the technique of FIG. 10 may be performed by a clinician, a user, and/or a patient.

A user may determine a first cuff position on a catheter (1002), remove a release liner of the first cuff (1004), wrap the first cuff around a perimeter of an outer surface of a catheter at the determined first cuff position (1006), and adhere the first cuff to the outer surface of the catheter (1008), e.g., as described with reference to blocks 902, 904, 906, and 908 of FIG. 9. The user may adhere the first cuff to the outer surface of the catheter (1008).

In the example of FIG. 10, the user also determined a second cuff position on a catheter (1010). In some examples, the user selects the second cuff position based on locations of exit site 328 and peritoneal cavity entrance site 338, e.g., such that the first cuff is positioned at exit site 328 and the second cuff is positioned at peritoneal cavity entrance site 338, or vice versa. For example, the user may additionally wish to avoid folds in skin 302 and/or the abdominal area of patient 12, and the user may then determine one or more desired exit sites 328 based on any patient 12 feature including avoiding skin folds. The user may then determine a length that catheter 314 should run within any and/or all of skin 302, subdermal tissue 204, and the abdominal wall 306 between peritoneal cavity entrance site 338 and exit site 328. The user may then determine the second cuff position (e.g., for a subcutaneous cuff 322) accordingly based on that determined length and the determined length of the distal portion of the catheter 314 for adequate and/or optimal PD, e.g., at (1004). In some examples, the user may determine the second cuff position using markings on the catheter, such as markings 202. The user may determine the second cuff position exactly on one or more marking 202, or the user may determine the second cuff position using markings 202 as a guide, e.g., for later positioning and attaching of the second cuff at the determined second cuff position.

The user may remove a release liner of the second cuff (1012). For example, the user may remove release liner 808 to expose adhesive 806, e.g. just prior to wrapping and adhering the second cuff 800 to the catheter.

The user may wrap the second cuff around a perimeter of an outer surface of catheter 314 at the determined second cuff position (1014). For example, the user may wrap the second cuff 800 at least partially around the perimeter of an outer surface of catheter 314, or the user may wrap the second cuff 800 fully around the perimeter of an outer surface of catheter 314 such that the ends of the second cuff 800 abut. In some examples, the user may wrap the second cuff 800 at least partially around, or fully around the perimeter of an outer surface of catheter 314 one or more times such that ends of the second cuff 800 do not abut, e.g., the user may wrap a helical cuff 800 (e.g., cuff 720 of FIGS. 7A and 7B) more than once around the perimeter of an outer surface of the catheter. In some examples, catheter 14 may include a plurality of markings along a length of the PD catheter body, and the user may wrap second cuff 800 around the perimeter of the outer surface of catheter 314 by aligning second cuff 800 with respective markings of the plurality of markings, e.g., markings 202.

The user may adhere the second cuff to the outer surface of the catheter (1016). For example, the user may apply pressure to the second cuff 800 that is wrapped around the perimeter of the outer surface of the catheter at the determined second cuff position. In some examples, the pressure may be a radially inward force that may cause adhesive 806 to adhere and/or increase adhesion to the outer surface of the catheter, e.g., adhesive 806 of the second cuff 800 may be a PSA that the user presses to the outer surface of the catheter.

In some examples, the user may perform the steps of the method in one or more different orders. For example, the clinician and or user may first determine the positioning of, and attach, the second cuff before the first cuff, e.g., the user and or clinician may perform (1010)-(1016) before (1002)-(1008).

Various examples of the disclosure have been described. Any combination of the described systems, operations, or functions is contemplated. These and other examples are within the scope of the following claims.

What is claimed is:

1. A catheter assembly comprising:
a catheter comprising an elongated catheter body defining a lumen;
a first cuff comprising:
a first cuff body comprising a material configured to promote epidermal tissue growth around or within the first cuff; and
an adhesive configured to adhere the first cuff body to an outer surface of the elongated catheter body at a first cuff position selectable by a user; and
a second cuff comprising:
a second cuff body comprising the material configured to promote epidermal tissue growth around or within the second cuff body; and
an adhesive configured to adhere the second cuff body to the outer surface of the elongated catheter body at a second cuff position selectable by the user,
wherein a distance between the first and second cuff positions is customizable by the user based on a patient.

2. The catheter assembly of claim 1, wherein the elongated catheter body comprises one or more markings along a length of the elongated catheter body, wherein the one or more markings indicate a plurality of different catheter cuff positions selectable by the user.

3. The catheter assembly of claim 1, wherein the first and second cuff positions are based on a determined length between a subcutaneous cuff position and a deep cuff position of the patient.

4. The catheter assembly of claim 1, wherein at least a distal portion of the elongated catheter body is coiled.

5. The catheter assembly of claim 1, wherein the first and second cuff bodies have at least one of a keyed shape or a helical shape.

6. The catheter assembly of claim 1, wherein the cuff body comprises an antimicrobial coating.

7. The catheter assembly of claim 1, wherein the adhesive is configured to adhere to at least one of silicone, urethane, or polyurethane.

8. The catheter assembly of claim 1, wherein the first and second cuff each further comprise a release liner, wherein the release liner is configured to releasably attach to the adhesive.

9. The catheter assembly of claim 1, wherein the first cuff body and the second cuff body each comprise a polyester fabric and a primer disposed on the polyester fabric, the primer configured to promote adhesion between the adhesive and the polyester fabric.

10. The catheter assembly of claim 1, wherein the adhesive is a pressure sensitive adhesive.

11. A catheter assembly comprising:
a peritoneal dialysis catheter comprising an elongated catheter body defining a lumen, a distal portion of the elongated catheter body defining a plurality of sidewall openings open to the lumen and configured to at least one of introduce fluid into a peritoneal cavity of a patient or remove fluid from the peritoneal cavity; and
a cuff comprising:
a cuff body comprising a material configured to promote epidermal tissue growth around or within the cuff; and
an adhesive configured to adhere the cuff body to an outer surface of the elongated catheter body at a cuff position selectable by a user.

12. The catheter assembly of claim 11, wherein the elongated catheter body comprises one or more markings along a length of the elongated catheter body, wherein the one or more markings indicate one or more of a plurality of different catheter cuff positions selectable by the user.

13. The catheter assembly of claim 11, wherein the cuff position is based on a determined length between a subcutaneous cuff position and a deep cuff position of the patient.

14. The catheter assembly of claim 11, wherein the cuff body has at least one of a keyed shape or a helical shape.

15. The catheter assembly of claim 11, wherein the cuff body comprises an antimicrobial coating.

16. The catheter assembly of claim 11, wherein the adhesive is configured to adhere to at least one of silicone, urethane, or polyurethane.

17. The catheter assembly of claim 11, wherein the cuff further comprises a release liner, wherein the release liner is configured to releasably attach to the adhesive.

18. A method of forming a peritoneal dialysis catheter, the method comprising:
determining a first cuff position on the peritoneal dialysis catheter;
wrapping a first cuff around a perimeter of an outer surface of the peritoneal dialysis catheter at the first cuff position, wherein the first cuff comprises:
a first cuff body comprising a material configured to promote epidermal tissue growth around or within the first cuff; and
an adhesive configured to adhere the first cuff body to an outer surface of the peritoneal dialysis catheter body at the first cuff position;
adhering, via applying pressure to the first cuff, the adhesive to the outer surface of the peritoneal dialysis catheter;
determining a second cuff position on the peritoneal dialysis catheter;
wrapping a second cuff around the perimeter of the outer surface of the peritoneal dialysis catheter at the second cuff position, wherein the second cuff comprises:
a second cuff body comprising a material configured to promote epidermal tissue growth around or within the second cuff; and
an adhesive configured to adhere the second cuff body to an outer surface of the peritoneal dialysis catheter body at the second cuff position; and
adhering, via applying pressure to the second cuff, the adhesive to the outer surface of the peritoneal dialysis catheter.

19. The method of claim 18, further comprising, before wrapping the first and second cuffs around the perimeter of the outer surface of the peritoneal dialysis catheter:
removing a first release liner of the first cuff, the first release liner covering the adhesive of the first cuff; and
removing a second release liner of the second cuff, the second release liner covering the adhesive of the second cuff.

20. The method of claim 18, wherein the peritoneal dialysis catheter comprises a plurality of markings along a length of the peritoneal dialysis catheter body, and wherein wrapping the first and second cuffs around the perimeter of the outer surface of the peritoneal dialysis catheter comprises aligning the first and second cuffs with respective markings of the plurality of markings.
